(19) United States
(12) Patent Application Publication
(10) Pub. No.: US 2024/0355423 A1
(43) Pub. Date: Oct. 24, 2024

(54) NUCLEIC ACID STRAND DETECTIONS

(71) Applicant: Hewlett-Packard Development Company, L.P., Spring, TX (US)

(72) Inventors: Yiming Zuo, Palo Alto, CA (US); Anton Wiranata, Boise, ID (US); Amy Devitt, Palo Alto, CA (US); Yang Lei, Palo Alto, CA (US); Steven James Barcelo, Palo Alto, CA (US); Brian John Keefe, San Diego, CA (US)

(73) Assignee: Hewlett-Packard Development Company, L.P., Spring, TX (US)

(21) Appl. No.: 18/684,138

(22) PCT Filed: Aug. 25, 2021
(86) PCT No.: PCT/US2021/047583

§ 371 Date: Feb. 15, 2024

(51) Int. Cl. G16B 40/10; C12Q 1/6844; G01N 21/64; G16B 40/20

(52) U.S. Cl. CPC G16B 40/10; C12Q 1/6844; G01N 21/6408; G16B 40/20; G01N 2201/1296

(57) ABSTRACT

Examples of methods are described herein. In some examples, a method includes determining signal variation data of a fluorescence signal measured from an amplification procedure of a nucleic acid sample. In some examples, the method includes detecting, using a machine learning model, a target nucleic acid strand in the nucleic acid sample based on the signal variation data.

### BACKGROUND

Nucleic acids are molecular structures made from polynucleotide chains, each containing a five-carbon sugar backbone, a phosphate group, and a nitrogen base. Ribonucleic acid (RNA) is a nucleic acid (e.g., molecular structure) that may include a single polynucleotide chain. Deoxyribonucleic acid (DNA) is a nucleic acid (e.g., molecular structure) including two polynucleotide chains that form a double helix. DNA and RNA include a sequence of nucleobase pairs (of four nucleobases cytosine, guanine, adenine, and thymine). For example, DNA includes nucleobases between sugar-phosphate backbones of the double helix. DNA and RNA serve as genetic instructions for the reproduction of organisms and viruses. Different organisms and viruses include different nucleic acid strands. For instance, different viruses may include different RNA strands.

### BRIEF DESCRIPTION OF THE DRAWINGS

FIG. 1 is a flow diagram illustrating an example of a method for target nucleic acid strand detection;

FIG. 2 is a block diagram illustrating an example of engines that may be utilized in accordance with some examples of the techniques described herein;

FIG. 3 is a block diagram of an example of an apparatus that may be used in nucleic acid strand detection;

FIG. 4 is a block diagram illustrating an example of a computer-readable medium for nucleic acid strand detection;

FIG. 5A is a graph illustrating examples of plots of positive pulse-controlled amplification (PCA) fluorescence signals and negative PCA fluorescence signals;

FIG. 5B is a graph illustrating an example of a plot of a positive PCA raw fluorescence signal and a smoothed fluorescence signal;

FIG. 6 is a graph illustrating examples of plots of positive nucleic acid samples and negative nucleic acid samples in a feature space; and

FIG. 7 is a graph illustrating examples of plots of positive nucleic acid samples and negative nucleic acid samples in a feature space.

### DETAILED DESCRIPTION

Examples of the techniques described herein provide approaches for the detection of a nucleic acid strand in a nucleic acid sample. A nucleic acid strand is a portion of DNA and/or RNA. A nucleic acid sample is biological material including a nucleic acid (e.g., DNA and/or RNA). Examples of nucleic acid samples include saliva, blood, mucus, sputum, urine, stool, cells, tissue, skin, etc.

An amplification procedure is a procedure to replicate or “amplify” a nucleic acid strand. Examples of amplification procedures include quantitative polymerase chain reaction (qPCR), pulse-controlled amplification (PCA), and reverse transcriptase polymerase chain reaction (RT-PCR). In an amplification procedure, a nucleic acid sample is repeatedly heated and cooled. Throughout heating and cooling cycles, measurements (e.g., fluorescence measurements) are taken.

In some examples of amplification procedures, a primer and/or fluorophore is added to a nucleic acid sample. A primer is a molecule that binds to a target nucleic acid strand (e.g., to a beginning and/or end of a target nucleic acid strand). For example, a nucleic acid sample may be heated (e.g., heated to 94° Celsius (C.)) or another temperature) to denature the nucleic acid (e.g., open the nucleobase pairs to expose the nucleobases). In some examples, after denaturing the nucleic acid, the nucleic acid sample may be cooled (e.g., cooled to between 50-60° (C.) or another temperature, annealed, etc.) and the primer may bind with the beginning and/or end of a target nucleic acid strand. In some examples, after cooling, the nucleic acid sample may be warmed (e.g., warmed to 72° C. or another temperature) and an enzyme (e.g., polymerase) may replicate the target nucleic acid strand (e.g., may add bases to the target nucleic acid strand from the primer binding site(s)). A fluorophore is a chemical compound that emits light after excitation. For example, a fluorophore may bond with a target nucleic acid strand. The nucleic acid sample may be excited with light. For example, a light emitting diode (LED), laser, or xenon lamp may be utilized to excite the nucleic acid sample with ultraviolet light and/or visible light, etc. The bonded fluorophore may emit light after excitation. The emitted light may be measured with a detector (e.g., light sensor, camera, etc.). For instance, the detector may produce a fluorescence measurement of the nucleic acid sample. Multiple cycles (e.g., denaturing, annealing, replication, and/or measurement) of the amplification procedure may be performed to create additional copies and measure an amount of the target nucleic acid strand in the nucleic acid sample.

Some examples of fluorophores may include 6-carboxyfluorescein (FAM), Cy5™, hexachlorofluorescein (HEX), and Texas Red (TEX). The wavelength of excitation light and/or emitted light utilized may vary in accordance with the fluorophore utilized. Examples of wavelengths for excitation light may include 495 nanometers (nm) for FAM, 648 nm for Cy5, 538 nm for HEX, and 596 nm for TEX. Examples of wavelengths for emitted light (e.g., detected light) may include 520 nm for FAM, 668 nm for Cy5, 555 nm for HEX, and 613 nm for TEX. In some examples, another fluorophore or fluorophores with a corresponding wavelength or wavelengths may be utilized. In some examples, the wavelength of excitation light provided and/or emitted light detected may vary from the examples given and/or may be performed over wavelength ranges. In some examples, one fluorophore with an excitation light wavelength (or wavelength range) and an emitted (e.g., detected) light wavelength (or wavelength range) may be utilized. In some examples, multiple (e.g., 2, 3, 4, 5, 6, etc.) fluorophores, excitation light wavelengths, and/or emitted light (e.g., detected light) wavelengths may be utilized. For instance, FAM and Cy5 fluorophores with corresponding wavelengths may be utilized. In some examples, multiple fluorescence measurements corresponding to respective fluorophores may be taken to produce multiple fluorescence signals (e.g., curves). In some examples, a fluorescence measurement for multiple fluorophores may be a sum, average, maximum, or other combination or selection of individual metrics (e.g., voltages, currents, etc.) for the respective fluorophores and/or wavelengths. In some examples, a single reaction chamber or multiple reaction chambers may be utilized in accordance with some examples of the techniques described herein.

PCA may be utilized to replicate and/or measure a target nucleic acid strand (e.g., target nucleic acids) from pathogens such as bacteria and viruses. For instance, PCA may be utilized to replicate and/or measure a target nucleic acid strand from Yersinia pestis for pneumonic plague or severe acute respiratory syndrome coronavirus 2 (SARS-COV-2) for COVID-19, etc. Relative to qPCR, PCA may reduce amplification time (e.g., approximately 1/10th of qPCR time) for rapid testing. For instance, PCA may utilize relatively rapid heating and cooling cycles. In some examples of PCA, a heating cycle may be performed on the order of microseconds or milliseconds (e.g., 5 microseconds (us), 15 μs, 50 μs, 100 μs, 200 μs, 0.5 milliseconds (ms), 1 ms, 2 ms, etc.) and/or may heat a portion of a nucleic acid sample. In some examples of PCA, cooling to annealing and/or extension temperatures may occur on the order of seconds (e.g., 1, 2, 3, 4, 5, 6 seconds, etc.). In some examples, a complete cycle (for heating, cooling, and/or measurement) may be completed on the order of seconds (e.g., 4, 5, 6, 10 seconds, etc.). In some examples, a PCA amplification procedure may take a few minutes (e.g., 7, 10, 15, 20 minutes, etc.) to complete. As a trade-off, PCA measurements (e.g., curves) may show an exponential shape with more embedded noise relative to a sigmoid shape of qPCR measurements (e.g., curves). Due to the exponential shape and/or increased noise in PCA measurements, it can be difficult to achieve the same level of sensitivity and specificity of qPCR. In some approaches, detection thresholds are manually set by experienced technicians. Manually setting detection thresholds may suffer from subjectivity, update complexity, and/or relatively long time delay for use in new applications.

In some examples of the techniques described herein, RT-PCR may be utilized to amplify RNA. For instance, a reverse transcriptase (RT) technique may be utilized to detect RNA via PCA.

Some examples of the techniques described herein may help detect a target nucleic acid strand in a nucleic acid sample using data-driven approaches. Some examples of the techniques described herein may be performed without manual threshold setting and/or may be utilized for relatively fast model updating for new applications.

Some examples of the techniques described herein may utilize a machine learning model or models to detect a target nucleic acid strand. A machine learning model is a structure that learns based on training. Examples of a machine learning model may include a regression model (e.g., regularized logistic regression models), a support vector machine (SVM), and an artificial neural network (e.g., deep neural networks, convolutional neural networks (CNNs), etc.). Training the machine learning model may include adjusting a weight or weights of the machine learning model. For example, a neural network may include a set of nodes, layers, and/or connections between nodes. The nodes, layers, and/or connections may have associated weights. The weights may be adjusted to train the neural network to perform a function, such as detecting a target nucleic acid strand based on fluorescence measurements.

Some examples of the techniques described herein utilize a feature or features of fluorescence measurements (e.g., PCA curve, qPCR curve, etc.). A feature is a metric that expresses a characteristic of a nucleic acid sample measurement. In some examples, a regularized logistic regression model or another machine learning model may be trained for target nucleic acid strand detection.

Throughout the drawings, similar reference numbers may designate similar or identical elements. When an element is referred to without a reference number, this may refer to the element generally, with and/or without limitation to any particular drawing or figure. In some examples, the drawings are not to scale and/or the size of some parts may be exaggerated to more clearly illustrate the example shown. Moreover, the drawings provide examples in accordance with the description. However, the description is not limited to the examples provided in the drawings.

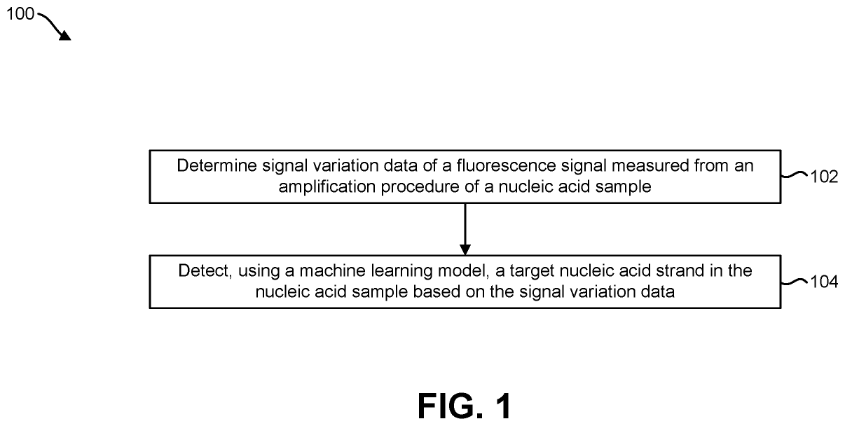

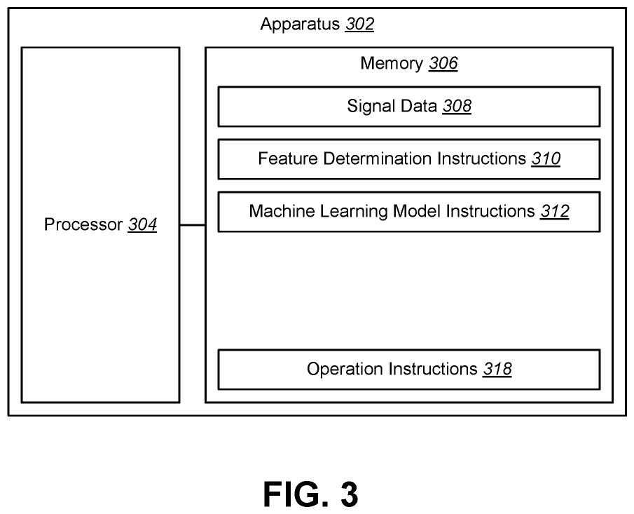

FIG. 1 is a flow diagram illustrating an example of a method 100 for target nucleic acid strand detection. The method 100 and/or an element or elements of the method 100 may be performed by an apparatus (e.g., electronic device). For example, the method 100 may be performed by the apparatus 302 described in connection with FIG. 3.

The apparatus may determine 102 signal variation data of a fluorescence signal measured from an amplification procedure of a nucleic acid sample. Signal variation data is data indicating a change in a signal. For example, signal variation data may indicate a change of a fluorescence signal over time.

In some examples, the method 100 may include truncating a portion of the fluorescence signal. For instance, measurements from an amplification procedure may be taken over a period of time (e.g., 10 minutes, 15 minutes, 30 minutes, 60 minutes, 90 minutes, etc.) to produce the fluorescence signal. In some examples, an initial portion (e.g., 0-30 seconds, 0-1 minute, 0-2 minutes, 0-5 minutes, etc.) of the fluorescence signal may be truncated (e.g., discarded). For instance, the first two minutes of a fluorescence signal (e.g., PCA curve) may be discarded due to increased noise in the first two minutes. In some examples, the signal variation data may be determined from the remaining (e.g., non-truncated) portion of the fluorescence signal.

In some examples, the method 100 may include smoothing the fluorescence signal to produce a smoothed fluorescence signal. For instance, the apparatus may calculate the smoothed fluorescence signal by computing a moving average (e.g., sliding window average, weighted moving average, etc.) of the fluorescence signal, low-pass filtering the fluorescence signal, and/or performing curve fitting (e.g., least-squares curve fitting) on the fluorescence signal, etc. Smoothing the fluorescence signal (e.g., the fluorescence signal after truncating a portion) may reduce high frequency noise. In some examples, determining 102 the signal variation data of a fluorescence signal as described herein may be based on the raw fluorescence signal and/or based on the smoothed fluorescence signal (with or without performing initial portion truncation, for instance).

In some examples, signal variation data may include or indicate a first change in a zeroth derivative of the fluorescence signal. A zeroth derivative of the fluorescence signal may be a fluorescence signal strength and/or a fluorescence signal amplitude. For instance, during the amplification procedure, the zeroth derivative of the fluorescence signal may be measured over time. In some examples, the zeroth derivative may be measured in volts (V), in current (e.g., amperes (A)), in relative fluorescence units, or in other units. For instance, a light emitter may excite the nucleic acid sample during the amplification procedure. A light sensor may sense and/or measure the fluorescence signal produced by the light sensor when sensing light (e.g., fluorescence) emitted by the nucleic acid sample. For instance, the light sensor may measure the fluorescence signal as a voltage amplitude, current amplitude, or as another metric. The zeroth derivative of the fluorescence signal may be an amplitude (e.g., volts) or strength of the fluorescence signal.

In some examples, the apparatus may determine the change in the zeroth derivative by determining a difference (e.g., subtraction) between values of the fluorescence signal at different times. For instance, the apparatus may subtract a value of an earlier portion of the fluorescence signal from a value of a later portion of the fluorescence signal. In some examples, the apparatus may determine a baseline zeroth derivative (from an earlier portion of the fluorescence signal, for instance). A “baseline” value is a value that represents a portion of a signal. For example, a baseline value may be determined from a portion (e.g., between 2-4 minutes) of the fluorescence signal. In some examples, A baseline zeroth derivative may be a value (e.g., average, mean, median, etc.) from a portion of the fluorescence signal. For instance, the apparatus may determine the baseline zeroth derivative as a median value in a 2-4 minute range of the smoothed fluorescence signal. In some examples, the change in the zeroth derivative of the fluorescence signal may be calculated by subtracting the last or final value of the smoothed fluorescence signal with the baseline zeroth derivative (e.g., subtracting the baseline zeroth derivative from the last or final value of the smoothed fluorescence signal). In some examples, the last or final value of the smoothed fluorescence signal may correspond to a last or final measurement of the amplification procedure (e.g., a final cycle of the amplification procedure).

In some examples, signal variation data may include or indicate a second change in a first derivative of the fluorescence signal. A first derivative of the fluorescence signal may be a slope of the fluorescence signal (e.g., smoothed fluorescence signal). The first derivative of the fluorescence signal may be an amplitude (e.g., volts) over a time of the fluorescence signal. In some examples, the apparatus may determine the slope of the fluorescence signal (e.g., smoothed fluorescence signal) for each measurement (e.g., at each measured time) of the fluorescence signal. For instance, the apparatus may determine an amplitude difference of values (e.g., amplitude difference within a time window, difference of adjacent values, etc.), over a difference in time (e.g., time increment) for each measurement of the fluorescence signal (e.g., smoothed fluorescence signal). In some examples, the apparatus may determine the slope as a difference of values at the beginning and end of a moving 1-minute time window (or another time window, for instance).

In some examples, the apparatus may determine the change in the first derivative by determining a difference (e.g., subtraction) between slopes of the fluorescence signal. For instance, the apparatus may subtract a slope from a portion of the fluorescence signal from a slope (e.g., maximum slope) of the fluorescence signal. In some examples, the apparatus may determine a baseline first derivative (from an earlier portion of the fluorescence signal, for instance). For instance, a baseline first derivative may be determined from a portion (e.g., between 2-4 minutes) of the fluorescence signal. In some examples, a baseline first derivative may be a slope (e.g., average slope, mean slope, median slope, etc.) from a portion of the fluorescence signal. For instance, the apparatus may determine the baseline first derivative as a median slope in a 2-4 minute range of the smoothed fluorescence signal. In some examples, the change in the first derivative of the fluorescence signal may be calculated by subtracting the maximum slope of the smoothed fluorescence signal with the baseline first derivative (e.g., subtracting the baseline first derivative from the maximum slope of the smoothed fluorescence signal).

In some examples, signal variation data may include or indicate a third change in a second derivative of the fluorescence signal. A second derivative of the fluorescence signal may be an acceleration of the fluorescence signal (e.g., smoothed fluorescence signal). The second derivative of the fluorescence signal may be an amplitude (e.g., volts) over a time squared of the fluorescence signal. In some examples, the apparatus may determine the acceleration of the fluorescence signal (e.g., smoothed fluorescence signal) for each measurement (e.g., at each measured time) of the fluorescence signal. For instance, the apparatus may determine a difference of slope values (e.g., slope difference within a time window, difference of adjacent slope values, etc.), over a difference in time (e.g., time increment) for each acceleration value (e.g., second derivative) of the fluorescence signal (e.g., smoothed fluorescence signal). In some examples, the apparatus may determine the acceleration as a difference of values at the beginning and end of a moving 1-minute time window (or another time window, for instance).

In some examples, the apparatus may determine the change in the second derivative by determining a difference (e.g., subtraction) between accelerations of the fluorescence signal. For instance, the apparatus may subtract an acceleration from a portion of the fluorescence signal from an acceleration (e.g., maximum acceleration) of the fluorescence signal. In some examples, the apparatus may determine a baseline second derivative (from an earlier portion of the fluorescence signal, for instance). For instance, a baseline second derivative may be determined from a portion (e.g., between 2-4 minutes) of the fluorescence signal. In some examples, a baseline second derivative may be an acceleration (e.g., average acceleration, mean acceleration, median acceleration, etc.) from a portion of the fluorescence signal. For instance, the apparatus may determine the baseline second derivative as a median acceleration in a 2-4 minute range of the smoothed fluorescence signal. In some examples, the change in the second derivative of the fluorescence signal may be calculated by subtracting the maximum acceleration of the smoothed fluorescence signal with the baseline second derivative (e.g., subtracting the baseline second derivative from the maximum acceleration of the smoothed fluorescence signal).

In some examples, the signal variation data include a first change in a zeroth derivative of the fluorescence signal, a second change in the first derivative of the fluorescence signal, and/or a third change in a second derivative of the fluorescence signal. In some examples, the method 100 may include determining a baseline zeroth derivative, a baseline first derivative, and a baseline second derivative of the fluorescence signal. In some examples, the method 100 may include determining the first change based on the baseline zeroth derivative, determining the second change based on the baseline first derivative, and/or determining the third change based on the baseline second derivative as described herein. In some examples, the first change in the zeroth derivative, the second change in the first derivative, and/or the third change in the second derivative may be a feature of features (e.g., features of a feature vector).

The apparatus may detect 104, using a machine learning model, a target nucleic acid strand in the nucleic acid sample based on the signal variation data. For example, the apparatus may input the signal variation data (e.g., the first change in the zeroth derivative, second change in the first derivative, and/or the third change in the second derivative) to the machine learning model. The machine learning model may detect whether the nucleic acid sample includes the target nucleic acid strand. For instance, the machine learning model may classify the nucleic acid sample based on the signal variation data and/or may infer whether the nucleic acid sample includes the target nucleic acid strand based on the signal variation data. The machine learning model may be trained to detect whether the target nucleic acid strand is in the nucleic acid sample.

In some examples, the machine learning model may be trained with labeled signal variation data. For example, the apparatus or another device may perform supervised training on the machine learning model. For instance, a training dataset may include features (e.g., individual features, feature vectors, changes in zeroth derivatives, changes in first derivatives, and/or changes in second derivatives, etc.) labeled to indicate whether the feature or features correspond to a nucleic acid sample that included the target nucleic acid strand. The weights of the machine learning model may be adjusted to reduce (e.g., minimize) classification error and/or to produce a decision boundary (e.g., decision hyperplane) that reduces (e.g., minimizes) misclassifications. In some examples, the machine learning model may be a regularized logistic regression model, an SVM model, an artificial neural network (e.g., CNN), or another machine learning model. Once the machine learning model is trained, the machine learning model may be executed to detect the target nucleic acid strand in the nucleic acid sample based on the signal variation data.

In some examples, the apparatus may perform an operation based on the detection. For instance, the apparatus may output an indicator (e.g., symbol, word, message, color, text, tone, sound, and/or speech, etc.) indicating whether the target nucleic acid strand was detected based on the signal variation data. In some examples, the apparatus may send an indicator to another device indicating whether the target nucleic acid strand was detected based on the signal variation data. For instance, the apparatus may send a message (e.g., packet(s), email, text message, phone call, alert, etc.) to another device (e.g., computer, smartphone, tablet device, and/or server, etc.) indicating whether the target nucleic acid strand was detected.

In some examples, the apparatus may perform the amplification procedure on the nucleic acid sample. For instance, the apparatus may include a reaction chamber. In some examples, the reaction chamber may include a heating element (e.g., heating plate, heating coil, etc.). The apparatus may control the heating element to cyclically heat the nucleic acid sample in the reaction chamber to a target temperature or temperatures. In some examples, the reaction chamber may include a light emitter and a light sensor. For instance, the apparatus may control the light emitter to cyclically emit light into the nucleic acid sample. The apparatus may take measurements from the light sensor. For instance, the apparatus may include an analog-to-digital converter (ADC) that samples voltages or currents taken from the light sensor. The measurements may be captured over a period as the fluorescence signal. The fluorescence signal may be utilized to determine 102 the signal variation data. In some examples, the amplification procedure is a PCA or qPCR amplification procedure.

Some examples of the techniques described herein may be performed for multiple fluorescence signals. For instance, an aspect or aspects of the method 100 may be performed for multiple fluorescence signals corresponding to respective (e.g., different) fluorophores. For example, respective fluorescence signals may be produced from an amplification procedure (e.g., PCA, qPCR, etc.). In some examples, a first fluorescence signal may correspond to a FAM fluorophore (e.g., E gene channel) and a second fluorescence signal may correspond to a Cy5 fluorophore (e.g., internal control channel). In some examples, a first fluorescence signal may indicate a sample measurement and a second fluorescence signal may indicate an internal process control measurement (which may be utilized to ensure that a reaction chamber is functioning correctly, for instance).

In some examples of the techniques described herein, the method 100 may include determining signal variation data for multiple fluorescence signals. For instance, the apparatus may determine respective first changes in zeroth derivatives of respective fluorescence signals, respective second changes in first derivatives of respective fluorescence signals, and/or respective third changes in second derivatives of respective fluorescence signals. In some examples of the techniques described herein, the method 100 may include detecting, using a machine learning model, a target nucleic acid strand in the nucleic acid sample based on the signal variation data for multiple fluorescence signals. For instance, the machine learning model may be trained to detect a target nucleic acid strand based on signal variation data (e.g., features) for multiple fluorescence signals. For instance, the machine learning model may detect a target nucleic acid strand based on a first fluorescence signal from FAM and a second fluorescence signal from Cy5.

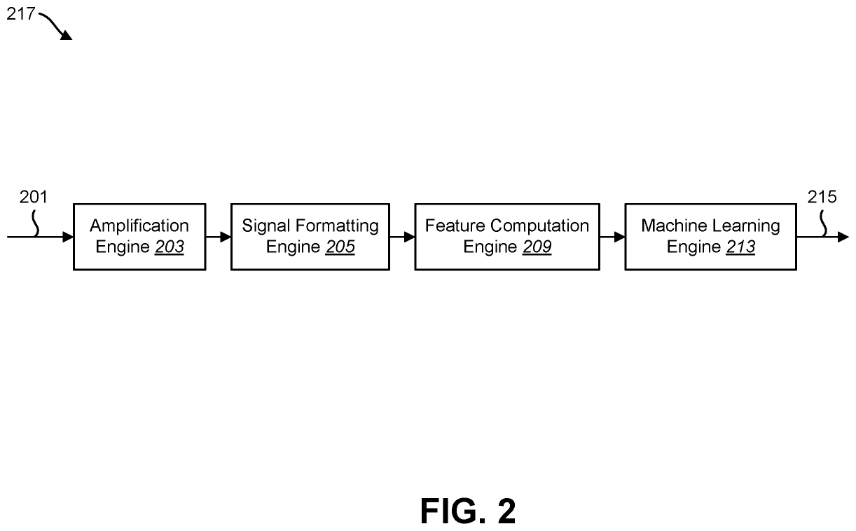

FIG. 2 is a block diagram illustrating an example of engines 217 that may be utilized in accordance with some examples of the techniques described herein. In some examples, an engine or engines of the engines 217 described in relation to FIG. 2 may be implemented in the apparatus 302 described in relation to FIG. 3. In some examples, a function or functions described in relation to any of FIGS. 1-7 may be implemented in an engine or engines described in relation to FIG. 2. An engine or engines described in relation to FIG. 2 may be implemented in a device or devices, in hardware (e.g., circuitry) and/or in a combination of hardware and instructions (e.g., processor and instructions). The engines described in relation to FIG. 2 include an amplification engine 203, signal formatting engine 205, a feature computation engine 209, and a machine learning engine 213. The engines 217 may be included in a same device or may be included in different devices in some examples. For instance, the amplification engine 203 may be included in a first device that may measure a fluorescence signal. The measured fluorescence signal may be provided to another device that includes the signal formatting engine 205, the feature computation engine 209, and the machine learning engine 213. In another examples, the amplification engine 203, the signal formatting engine 205, the feature computation engine 209, and the machine learning engine 213 may be included in one device.

In some examples of the techniques described herein, a nucleic acid sample 201 may be provided to the amplification engine 203. For instance, a technician may pipette the nucleic acid sample (with primer and fluorophore, for instance) into a reaction chamber of the amplification engine 203. The amplification engine 203 may perform an amplification procedure (e.g., PCA or qPCR) and measure a fluorescence signal as described herein. The fluorescence signal may be provided to the signal formatting engine 205.

The signal formatting engine 205 may format the fluorescence signal. For instance, the signal formatting engine 205 may truncate and smooth the fluorescence signal as described in relation to FIG. 1. The truncated and smoothed fluorescence signal may be provided to the feature computation engine 209. The feature computation engine 209 may compute a feature or features based on the fluorescence signal (e.g., truncated and smoothed fluorescence signal). For instance, the feature computation engine 209 may compute a first change in a zeroth derivative of the fluorescence signal, a second change in a first derivative of the fluorescence signal, and/or a third change in a second derivative of the fluorescence signal. The feature(s) may be provided to the machine learning engine 213.

The machine learning engine 213 may determine, using a machine learning model, whether the nucleic acid sample 201 includes a target nucleic acid strand based on the feature(s). For instance, the machine learning engine 213 may classify the nucleic acid sample 201 as including the target nucleic acid strand or not based on the feature(s) as described in relation to FIG. 1. The machine learning engine 213 may produce an indicator 215 (e.g., message, number, text, etc.) indicating whether the nucleic acid sample 201 includes a target nucleic acid strand. For instance, the indicator 215 may be displayed, used to produce an output, and/or sent to another device to indicate whether the nucleic acid sample 201 includes the target nucleic acid strand.

FIG. 3 is a block diagram of an example of an apparatus 302 that may be used in nucleic acid strand detection. The apparatus 302 may be a computing device, such as a personal computer, a server computer, a smartphone, a tablet computer, an electronic diagnostic device, an electronic testing device, a mobile testing device, a handheld electronic device, etc. The apparatus 302 may include and/or may be coupled to a processor 304 and/or a memory 306. The processor 304 may be in electronic communication with the memory 306. In some examples, the apparatus 302 may be in communication with (e.g., coupled to, have a communication link with) another device or devices (e.g., reaction chamber, nucleic acid amplification device, PCA device, server, computer, smartphone, tablet device, etc.). In some examples, the apparatus 302 may be an example of a computer. In some examples, the apparatus 302 may be an example of a medical testing device. The apparatus 302 may include additional components (not shown) and/or some of the components described herein may be removed and/or modified without departing from the scope of this disclosure.

In some examples, the apparatus 302 may perform a technique or techniques (e.g., measurement, signal variation data determination, feature computation, and/or detection, etc.) described herein without sending data to another device and/or without receiving data from another device (e.g., a cloud server, an edge device, a networked device, etc.). For instance, the apparatus 302 may be a local medical testing device and/or computer, where a communication bus and/or network interface is not used to send and/or receive data pertaining to some examples of the techniques described herein (e.g., measurement, signal variation data determination, feature computation, and/or detection). In some examples, a technique or techniques (e.g., measurement, signal variation data determination, feature computation, and/or detection, etc.) described herein may be performed in conjunction with sending data to another device and/or receiving data from another device (e.g., a cloud server, an edge device, a networked device, etc.). For instance, the apparatus 302 may be a local medical testing device and/or computer that sends fluorescence signal(s) and/or feature(s) to a cloud server to perform a technique or techniques described herein (e.g., signal variation data determination, feature computation, and/or detection) and receives data (e.g., test results) from the cloud server. In some examples, the apparatus 302 may be a cloud server or edge device that receives data (e.g., fluorescence signal(s), signal variation data, and/or feature data) from another device, performs signal variation data determination, feature computation, and/or detection, and sends data (e.g., results) to another device (e.g., endpoint node).

The processor 304 may be any of a central processing unit (CPU), a semiconductor-based microprocessor, graphics processing unit (GPU), field-programmable gate array (FPGA), an application-specific integrated circuit (ASIC), and/or other hardware device suitable for retrieval and execution of instructions stored in the memory 306. The processor 304 may fetch, decode, and/or execute instructions (e.g., feature determination instructions 310, machine learning model instructions 312, and/or operation instructions 318) stored in the memory 306. In some examples, the processor 304 may include an electronic circuit or circuits that include electronic components for performing a functionality or functionalities of the instructions (e.g., feature determination instructions 310, machine learning model instructions 312, and/or operation instructions 318). In some examples, the processor 304 may perform one, some, or all of the functions, operations, elements, methods, etc., described in connection with one, some, or all of FIGS. 1-7.

The memory 306 may be any electronic, magnetic, optical, or other physical storage device that contains or stores electronic information (e.g., instructions and/or data). Thus, the memory 306 may be, for example, Random Access Memory (RAM), Electrically Erasable Programmable Read-Only Memory (EEPROM), a storage device, an optical disc, and the like. In some implementations, the memory 306 may be a non-transitory tangible machine-readable storage medium, where the term “non-transitory” does not encompass transitory propagating signals.

In some examples, the apparatus 302 may also include a data store (not shown) on which the processor 304 may store information. The data store may be volatile and/or non-volatile memory, such as Dynamic Random-Access Memory (DRAM), EEPROM, magnetoresistive random-access memory (MRAM), phase change RAM (PCRAM), memristor, flash memory, and the like. In some examples, the memory 306 may be included in the data store. In some examples, the memory 306 may be separate from the data store. In some approaches, the data store may store similar instructions and/or data as that stored by the memory 306. For example, the data store may be non-volatile memory and the memory 306 may be volatile memory.

In some examples, the apparatus 302 may include an input/output interface (not shown) through which the processor 304 may communicate with an external device or devices (not shown), for instance, to send and/or receive data. The input/output interface may include hardware and/or machine-readable instructions to enable the processor 304 to communicate with the external device or devices. The input/output interface may enable a wired and/or wireless connection to the external device or devices. In some examples, the input/output interface may further include a network interface card and/or may also include hardware and/or machine-readable instructions to enable the processor 304 to communicate with various input and/or output devices, such as a keyboard, a mouse, a display, touch screen, another apparatus, electronic device, computing device, etc., through which a user may input instructions into the apparatus 302. In some examples, the apparatus 302 may receive signal data 308 from an external device or devices (e.g., reaction chamber, testing device, etc.). For instance, the apparatus 302 may receive signal data 308 that indicates a fluorescence signal measured from an amplification procedure performed by a separate reaction chamber.

In some examples, the memory 306 may store signal data 308. Some examples of signal data 308 include data representing a fluorescence signal measured from an amplification procedure. The signal data 308 may be measured by the apparatus 302 and/or received from another device. For instance, the apparatus 302 may include a reaction chamber in some examples. The apparatus 302 (e.g., processor 304) may control the reaction chamber to perform an amplification procedure (on a nucleic acid sample, for instance). The apparatus 302 (e.g., processor 304) may measure the fluorescence signal (from the reaction chamber, for instance). For example, the processor 304 may control a reaction chamber to cyclically heat the nucleic acid sample, to emit light into the nucleic acid sample, and to measure fluorescence emitted from the nucleic acid sample. The measured fluorescence may be stored in the signal data 308 as a fluorescence signal.

The memory 306 may store feature determination instructions 310. The processor 304 may execute the feature determination instructions 310 to determine a feature or features based on the fluorescence signal represented by the signal data 308. In some examples, the processor 304 may determine a feature or features (e.g., first change in a zeroth derivative, second change in a first derivative, and/or third change in a second derivative, etc.) as described in relation to FIG. 1 and/or FIG. 2.

In some examples, the processor 304 may execute the feature determination instructions 310 to determine a slope curve of a fluorescence signal measured from an amplification procedure of a nucleic acid sample. For instance, a slope curve may be determined over the fluorescence signal as described in relation to FIG. 1.

The slope curve may be utilized to determine a feature. For instance, the processor 304 may execute the feature determination instructions 310 to compute a slope change based on the slope curve. In some examples, the slope change may be computed as a difference between a baseline slope (e.g., baseline first derivative) and a maximum slope (e.g., maximum first derivative) of the fluorescence signal. For instance, the processor 304 may discard a first portion of the fluorescence signal and smooth the fluorescence signal to produce a smoothed fluorescence signal. In some examples, the processor 304 may determine a baseline slope from a second portion of the smoothed fluorescence signal and determine a maximum slope of the smoothed signal. Computing the slope change may include determining a difference between the baseline slope and the maximum slope.

The memory 306 may store machine learning model instructions 312. The processor 304 may execute the machine learning model instructions 312 to determine, using a machine learning model, whether the nucleic acid sample includes a target nucleic acid strand based on the feature(s). For instance, the processor 304 may execute a machine learning model that is trained based on the feature or features to detect the target nucleic acid strand. In some examples, the machine learning model may detect the target nucleic acid strand in the nucleic acid sample as described in relation to FIG. 1. In some examples, the processor 304 may execute the machine learning model instructions 312 to determine, using a machine learning model, whether the nucleic acid sample includes a target nucleic acid strand based on the slope change.

In some examples, the processor 304 may execute the operation instructions 318 to perform an operation. For example, the apparatus 302 may perform an operation based on the determination of whether the nucleic acid sample includes the target nucleic acid strand. For instance, the apparatus 302 may output an indicator (e.g., symbol, word, message, color, text, tone, sound, and/or speech, etc.) indicating whether the target nucleic acid strand was detected based on the signal variation data. In some examples, the apparatus 302 may send an indicator to another device indicating whether the target nucleic acid strand was detected based on the signal variation data. For instance, the apparatus 302 may be a server that receives a fluorescence signal from another device and provides a testing web service. The apparatus 302 may send a message (e.g., packet(s), email, text message, phone call, alert, etc.) to another device (e.g., computer, smartphone, tablet device, and/or server, etc.) indicating whether the target nucleic acid strand was detected. For instance, the apparatus 302 may send a message to a requesting device indicating whether the target nucleic acid strand was detected. In some examples, the apparatus 302 may send a message to another device (e.g., server) to report a number of cases in which the target nucleic acid strand was detected.

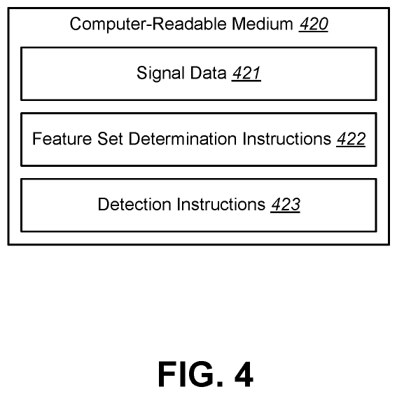

FIG. 4 is a block diagram illustrating an example of a computer-readable medium 420 for nucleic acid strand detection. The computer-readable medium 420 may be a non-transitory, tangible computer-readable medium 420. The computer-readable medium 420 may be, for example, RAM, EEPROM, a storage device, an optical disc, and the like. In some examples, the computer-readable medium 420 may be volatile and/or non-volatile memory, such as DRAM, EEPROM, MRAM, PCRAM, memristor, flash memory, and the like. In some implementations, the memory 306 described in connection with FIG. 3 may be an example of the computer-readable medium 420 described in connection with FIG. 4.

The computer-readable medium 420 may include data (e.g., information and/or instructions). For example, the computer-readable medium 420 may include signal data 421, feature set determination instructions 422, and/or detection instructions 423.

In some examples, the computer-readable medium 420 may store signal data 421. Some examples of signal data 421 include data representing a fluorescence signal or signals, signal variation data, signal feature data, etc. For instance, the signal data 421 may represent a fluorescence signal or signals measured from an amplification (e.g., PCA, qPCR, etc.) procedure.

In some examples, the feature set determination instructions 422 may be instructions when executed cause a processor of an electronic device to determine a feature set based on a fluorescence signal measured from a PCA procedure. In some examples, determining the feature set may be accomplished as described in relation to FIG. 1.

In some examples, the feature set determination instructions 422 may include instructions when executed cause the processor to determine a difference between a baseline signal strength (e.g., baseline zeroth derivative) and a final signal strength (e.g., last value measured from the PCA procedure). For instance, determining the difference between the baseline signal strength and the final signal strength may produce a signal strength change in the feature set.

In some examples, the feature set determination instructions 422 may include instructions when executed cause the processor to determine a difference between a baseline signal slope (e.g., baseline first derivative of the fluorescence signal) and a maximum signal slope (e.g., maximum first derivative of the fluorescence signal). For instance, determining the difference between the baseline signal slope and the maximum signal slope may produce a maximum signal slope in the feature set.

In some examples, the feature set determination instructions 422 may include instructions when executed cause the processor to determine an acceleration feature (e.g., feature from the second derivative of the fluorescence signal). For instance, the acceleration feature may be determined for the feature set.

In some examples, the detection instructions 423 may be instructions when executed cause the processor to execute a machine learning model to detect a target nucleic acid strand in a nucleic acid sample based on the feature set. In some examples, detecting the target nucleic acid strand may be accomplished as described in relation to FIG. 1, FIG. 2, and/or FIG. 3.

In some examples, the computer-readable medium 420 may include instructions when executed cause the processor to train the machine learning model. In some examples, this may be accomplished as described in relation to FIG. 1.

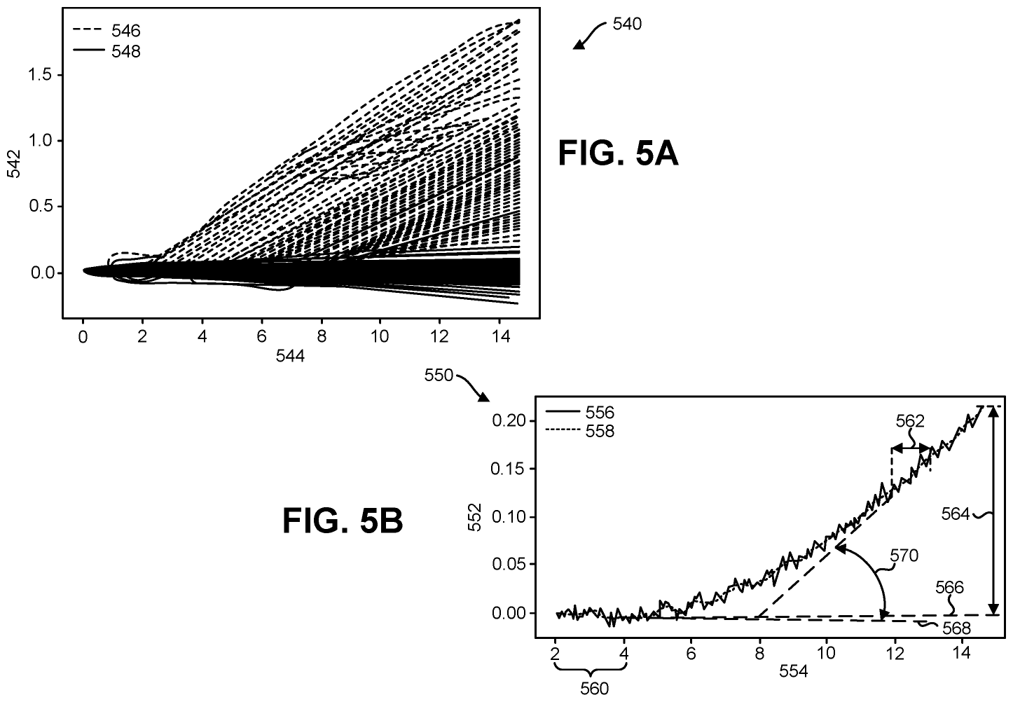

FIG. 5A is a graph 540 illustrating examples of plots of positive PCA fluorescence signals 546 and negative PCA fluorescence signals 548. For example, the graph 540 illustrates the plots in fluorescence signal change 542 (in volts) over time 544 (in minutes). The positive PCA fluorescence signals 546 are from nucleic acid samples in which a target nucleic acid strand was present, while the negative PCA fluorescence signals 548 are from nucleic acid samples in which the target nucleic acid strand was not present. In these examples, the PCA fluorescence signals are from nucleic acid samples with different viral loads. The positive PCA fluorescence signals 546 have exponentially shaped curves, which may differ from sigmoid-shaped qPCR curves. It may be difficult to set up a threshold purely based on a fluorescence signal to separate positive and negative groups for PCA fluorescence signals.

FIG. 5B is a graph 550 illustrating an example of a plot of a positive PCA raw fluorescence signal 556 and a smoothed fluorescence signal 558. For example, the graph 550 illustrates the plots in fluorescence signal change 552 (in volts) over time 554 (in minutes). As illustrated in the graph 550, the raw fluorescence signal 556 may be truncated, where a portion of the raw fluorescence signal 556 from 0-2 minutes has been removed. Smoothing the raw fluorescence signal 556 may produce the smoothed fluorescence signal 558.

In the example of FIG. 5B, a baseline period 560 (from 2-4 seconds) of the smoothed fluorescence signal 558 may be utilized to determine a baseline signal strength 566. For example, the median signal strength of the smoothed fluorescence signal 558 may be determined as the baseline signal strength 566. A signal strength change 564 (e.g., change in the zeroth derivative) may be calculated by subtracting the baseline signal strength 566 and the final value of the smoothed fluorescence signal 558.

In the example of FIG. 5B, a 1-minute window 562 (e.g., a sliding window) may be utilized to determine slope values over the smoothed fluorescence signal 558. A median slope value within the baseline period 560 may be determined as a baseline slope 568. A slope change 570 (e.g., change in the first derivative) may be calculated by subtracting the baseline slope 568 and the maximum slope. As described herein, the signal strength change 564, the slope change 570, and/or another value(s) (e.g., acceleration change) may be utilized as features to detect a target nucleic acid strand.

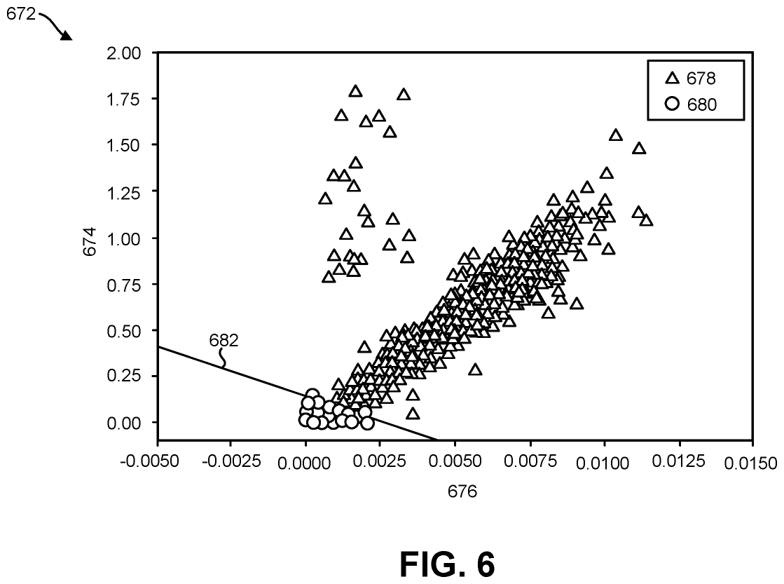

FIG. 6 is a graph 672 illustrating examples of plots of positive nucleic acid samples 678 and negative nucleic acid samples 680 in a feature space. For example, the graph 672 illustrates the plots in fluorescence signal change 674 (in volts) over slope change 676 (in volts over time). The positive nucleic acid samples 678 are nucleic acid samples in which a target nucleic acid strand was present, while the negative nucleic acid samples 680 are nucleic acid samples in which the target nucleic acid strand was not present. In this example, a regularized logistic regression model is trained to produce a decision boundary 682 in the feature space. Accordingly, the regularized logistic regression model may be utilized to detect positive nucleic acid samples 678 based on examples of the features described herein (e.g., signal strength change and slope change).

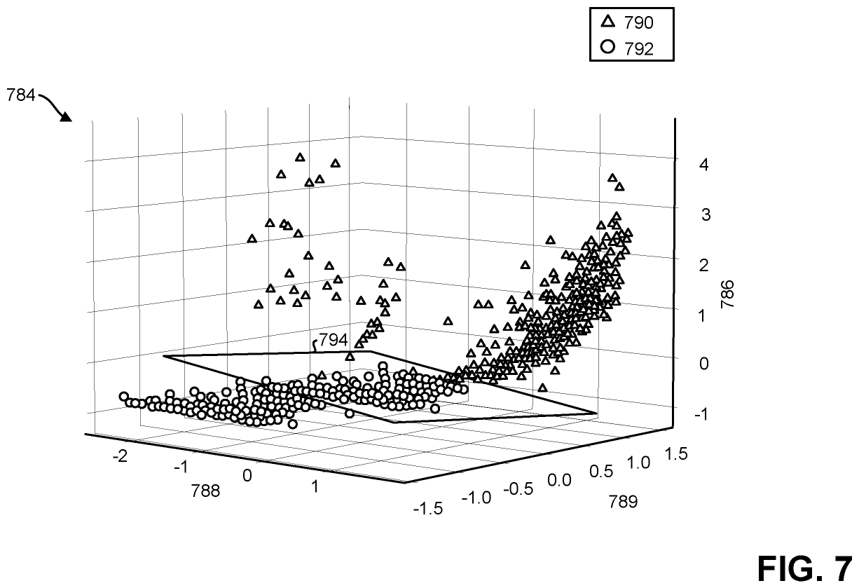

FIG. 7 is a graph 784 illustrating examples of plots of positive nucleic acid samples 790 and negative nucleic acid samples 792 in a feature space. For example, the graph 784 illustrates the plots in a zeroth-order derivative feature 786 (e.g., fluorescence signal change (in volts)) a first-order derivative feature 788 (e.g., slope change (in volts over time)) over a second-order derivative feature 789 (e.g., acceleration change (in volts over time squared)). The positive nucleic acid samples 790 are nucleic acid samples in which a target nucleic acid strand was present, while the negative nucleic acid samples 792 are nucleic acid samples in which the target nucleic acid strand was not present. In this example, a machine learning model is trained to produce a decision hyperplane 794 in the feature space. Accordingly, the machine learning model may be utilized to detect positive nucleic acid samples 790 based on examples of the features described herein (e.g., signal strength change, slope change, and acceleration change).

Some examples of the techniques described herein may provide detection approaches that are data-driven and interpretable. For instance, the data-driven detection results may be explainable and transparent (to regulator(s) and/or user(s), for instance). For example, the functioning of a data-driven machine learning model may be interpretable, such that the reasons for a detection result being produced are explainable and/or transparent.

As used herein, the term “and/or” may mean an item or items. For example, the phrase “A, B, and/or C” may mean any of: A (without B and C), B (without A and C), C (without A and B), A and B (but not C), B and C (but not A), A and C (but not B), or all of A, B, and C.

While various examples of systems and methods are described herein, the systems and methods are not limited to the examples. Variations of the examples described herein may be implemented within the scope of the disclosure. For example, operations, functions, aspects, or elements of the examples described herein may be omitted or combined.

1. A method, comprising:
determining signal variation data of a fluorescence signal measured from an amplification procedure of a nucleic acid sample; and
detecting, using a machine learning model, a target nucleic acid strand in the nucleic acid sample based on the signal variation data.

2. The method of claim 1, wherein the signal variation data comprises a first change in a zeroth derivative of the fluorescence signal and a second change in a first derivative of the fluorescence signal.

3. The method of claim 2, wherein the signal variation data further comprises a third change in a second derivative of the fluorescence signal.

4. The method of claim 3, further comprising:
determining a baseline zeroth derivative, a baseline first derivative, and a baseline second derivative of the fluorescence signal; and
determining the first change based on the baseline zeroth derivative;
determining the second change based on the baseline first derivative; and
determining the third change based on the baseline second derivative.

5. The method of claim 1, further comprising truncating a portion of the fluorescence signal.

6. The method of claim 1, further comprising performing the amplification procedure on the nucleic acid sample.

7. The method of claim 6, wherein the amplification procedure is pulse-controlled amplification (PCA) procedure.

8. The method of claim 1, wherein the machine learning model is a regularized logistic regression model, a support vector machine (SVM) model, or an artificial neural network.

9. The method of claim 1, wherein the machine learning model is trained with labeled signal variation data.

10. An apparatus, comprising:
a memory;
a processor in electronic communication with the memory, wherein the processor is to:
determine a slope curve of a fluorescence signal measured from an amplification procedure of a nucleic acid sample;
compute a slope change based on the slope curve; and
determine, using a machine learning model, whether the nucleic acid sample includes a target nucleic acid strand based on the slope change.

11. The apparatus of claim 10, wherein the processor is to:
discard a first portion of the fluorescence signal;
smooth the fluorescence signal to produce a smoothed fluorescence signal;
determine a baseline slope from a second portion of the smoothed fluorescence signal; and
determine a maximum slope of the smoothed fluorescence signal, wherein computing the slope change comprises determining a difference between the baseline slope and the maximum slope.

12. The apparatus of claim 11, further comprising:
a reaction chamber, wherein the processor is to:
control the reaction chamber to perform the amplification procedure; and
measure the fluorescence signal.

13. A non-transitory tangible computer-readable medium comprising instructions when executed cause a processor of an electronic device to:
determine a feature set based on a fluorescence signal measured from a pulse-controlled amplification (PCA) procedure; and
execute a machine learning model to detect a target nucleic acid strand in a nucleic acid sample based on the feature set.

14. The non-transitory tangible computer-readable medium of claim 13, further comprising instructions when executed cause the processor to determine a difference between a baseline signal strength and a final signal strength.

15. The non-transitory tangible computer-readable medium of claim 13, further comprising instructions when executed cause the processor of the electronic device to determine a difference between a baseline signal slope and a maximum signal slope.
